(19) United States
(12) Patent Application Publication
(10) Pub. No.: US 2015/0269325 A1
(43) Pub. Date: Sep. 24, 2015

(54) MEDICAL TEST RESULT DISPLAY DEVICE AND METHOD FOR OPERATING THE SAME

(71) Applicant: FUJIFILM CORPORATION, TOKYO (JP)

(72) Inventors: YASUNORI OHTA, KANAGAWA (JP); SHOJI KANADA, KANAGAWA (JP)

(21) Appl. No.: 14/724,835

(22) Filed: May 29, 2015

Related U.S. Application Data
(63) Continuation of application No. PCT/JP2013/081867, filed on Nov. 27, 2013.

(30) Foreign Application Priority Data
Nov. 30, 2012 (JP) 2012-262926

(51) Int. Cl. G06F 19/00; G06T 11/20; G06F 3/0482; G06T 11/60; A61B 5/00; G06F 3/0484

(52) U.S. Cl. CPC G06F 19/322; A61B 5/743; G06F 3/04842; G06F 3/0482; G06T 11/60; G06T 11/206; G06F 19/321; G06T 2200/24; G06T 2210/41

(57) ABSTRACT

A medical test result display device is provided, which enables checking test values of important test items determined according to the test purpose without the need for cumbersome operations. A test result collection and integration server collects medical information and medical images of a designated patient from a medical record storage server, and produces a test result display screen that integrates the medical information and images. When a thumbnail image of one medical image is selected on the test result display screen, a test purpose is retrieved from attached information of the corresponding medical image. Furthermore, important test items corresponding to the test purpose are retrieved from important test item information, thereby to revise the test result display screen to display one test values of the important test items.

### CROSS-REFERENCE TO RELATED APPLICATIONS

This application is a Continuation of PCT International Application No. PCT/JP2013/081867 filed on Nov. 27, 2013, which claims priority under 35 U.S.C §119(a) to Japanese Patent Application No. 2012-262926 filed Nov. 30, 2012. The above application is hereby expressly incorporated by reference, in its entirety, into the present application.

### BACKGROUND OF THE INVENTION

1. Field of the Invention

The present invention relates to a medical test result display device and a method for operating the same.

2. Description Related to the Prior Art

In medical institutions, such as hospitals, computer systems are increasingly introduced for storing and managing medical information created during the medical practices on patients as electronic data, the medical information including medical examination records, medical treatment records, medical test results, etc. As one of these computer systems, a medical test result display device (hereinafter referred to simply as test result display device), which displays results of medical tests carried out on patients, has been known (refer to Japanese Laid-open Patent Applications Nos. 2010-097476 and 2012-038349).

There are various kinds of medical tests, which may be roughly categorized into imaging tests, wherein medical images, such as X-ray images or ultrasonic images, are captured to serve as the test result, and those tests of which the test results are expressed by numerical test values, such as vital examination and blood test. Hereinafter, those medical tests of which the test results are expressed by numerical test values will be expedientially referred to as clinical tests for the sake of distinction from the imaging test. The clinical tests often include multiple test items in one test. Take a vital examination for instance, the test items thereof may include pulse, blood pressure, body temperature, etc. As for a blood test, the test items thereof may include white blood cell count, red blood cell count, hemoglobin concentration in blood, etc.

Test result display devices disclosed in Japanese Laid-open Patent Applications Nos. 2010-097476 and 2012-038349 have a capability of displaying test results of a plurality of tests, which have been carried out at different time periods, in the form of time-series graphs of test values on individual test items. For instance, the test result display device creates a time-series graph of test values on each test item, such as pulse or blood pressure for the vital examination, or white blood cell count or red blood cell count for the blood test, and displays the graphs on a display screen. The time-series graph of test values shows a temporal change in test values, facilitating recognizing the stage or extent of injury or disease in a patient and the therapeutic effects, such as the postoperative course and the reactions of medication, as well. Since the extent of injury or disease and the therapeutic effects provide important indicia for deciding therapeutic strategy, the time-series graph of test values is very useful.

In addition, the test result display devices in Japanese Laid-open Patent Applications Nos. 2010-097476 and 2012-038349 have a capability of displaying medical images, which have been captured for imaging tests, in a time-series fashion in addition to the time-series graph of test values. Medical images of a patient and test values on the patient are associated with each other by means of a patient ID that is information for identifying the patient, so that it is possible to read out the medical images or the test values on the basis of the patient ID while the test values or the medical images are being displayed on the display screen, respectively. Since both medical images and test values are often referred to during the medical practices, the capability of retrieving medical images on the basis of the associated test values and vice versa is also very useful.

However, the test result display device in Japanese Laid-open Patent Applications No. 2010-097476 has a problem in that the operation thereof becomes cumbersome as the number of kinds of tests or test items increases, because of the following reasons. In general, medical practice are carried out in the following sequence. First, clinical examinations of a patient through interviews and the like are conducted to narrow down to a suspected injury or disease, and various kinds of tests are made on the patient in order to determine the injury or disease. From the comprehensive diagnoses based on the test results, the injury or disease is determined. If more than one injury or disease is suspected from the interview, or the determined injury or disease is likely to accompany another injury or disease, many kinds of tests will be conducted with respect to the multiple injuries or diseases. After determining the injury or disease, a doctor decides on a therapeutic strategy on the basis of the test results, and provides medical treatments on the patient while repeating the medical tests to check the therapeutic effects.

In a medical institution, an inspection department is divided from clinical departments, such as a surgeon department and an internist department, which are mainly in charge of clinical examination and treatment. The inspection department is for conducting a test in accordance with an order for the test that is issued at any of the clinical departments. When a clinical test, such as a vital examination or a blood test, is ordered, the inspection department provides test values of a plurality of test items included in the clinical test in the form of a list, and forwards the list as test results to the clinical department.

When a doctor in a clinical department suspects a particular injury or disease, the doctor provides medical care focusing on particular important test items among multiple test items included in the test results from the patient. The important test items are those test items which the doctor must check up as particularly important items for the medical practice on the injury or disease. Since the important test items are different between different kinds of injuries and diseases, the important test items should be selected according to the injuries and diseases. Needless to say, the important test items change according to the kinds of clinical tests. For example, from among multiple test items for a blood test, a doctor in a clinical department selects important test items according to the injury and disease, and checks the consequent test values. Likewise, if it is necessary to refer to test results of a vital examination in addition to the blood test, the doctor selects important test items from among multiple test items for the vital examination according to the injury or disease, and checks the consequent test values. Thus, during the medical practice, multiple kinds of tests are repeated while making a selecting operation to select important test items according to the injury or disease from among multiple test items.

As for imaging tests, the clinical departments often issue an order for test while designating a test purpose, and the inspection department captures images in a method variable according to the test purpose. The test purpose clearly indicates a designated injury or disease, e.g., the purpose is for diagnosing lung cancer, pneumonia, stomach cancer, etc. The captured medical images are stored in association with the test order including the test purpose.

The test result display devices in Japanese Laid-open Patent Applications Nos. 2010-097476 and 2012-038349 makes it easy to browse both medical images and test results with respect to a patient on a test result display screen, since the medical images and the test values are correlated with an ID of the patient. However, in a case where a wide variety of kinds of tests have been carried out a lot of times in the course of medical practices on a patient, a large number of test values and medical images will be displayed on a display screen. If the patient is suspected to contract more than one injury or disease, or suffers from multiple injuries and diseases, the number of test values and the number of medical images will further increase. Since medical images are captured on the basis of the test purpose for designated an injury or disease, if test values of important test items relating to a medical image are wanted to be observed with the medical image on the display screen, it is necessary to select the important test items for each medical image; thus, the operation will be so complicated that it has been a particular problem.

### SUMMARY OF THE INVENTION

The present invention has an object to provide a medical test result display device and a method for operating same, which make it easy to check test values of important test items appropriate to the test purpose without any cumbersome operation.

In order to solve the above problem, a medical test result display device in accordance with the present invention comprises a test results reading section, a test purpose reading section, a screen production unit, an important test item reading section and an image select information accepting section. The test results reading section accesses a medical image storage and a test results storage to read out medical images and test values of multiple test items. The medical image storage stores medical images captured at imaging tests carried out on a patient, each imaging test having at least a test purpose. The test result storage stores numerical test values that represent test results of multiple test items included in clinical tests carried out on the patient. The test purpose reading section accesses a test purpose storage to read out at least a test purpose, wherein the test purpose storage stores respective test purposes of the imaging tests. The screen production unit produces a test result display screen having an image display zone in which multiple medical images are capable of being displayed and a test value display zone in which test values of the multiple test items are capable of being displayed. The important test item reading section accesses an important test item storage to read out important test items according to a test purpose, wherein the important test item storage stores important test items selected for each test purpose from among the multiple test items. The image select information accepting section accepts image select information indicating that at least a medical image is selected from among the multiple medical images displayed in the image display zone. Furthermore, the screen production unit retrieves a test purpose corresponding to the selected medical image through the test purpose reading section on the basis of the image select information, retrieves important test items through the important test item reading section on the basis of the retrieved test purpose, and lets merely test values of the important test items be displayed in the test value display zone on the basis of the retrieved important test items.

Preferably, an injury or disease is designated in the test purpose.

When multiple medical images are selected on the test result display screen, the screen production unit preferably retrieves respective test purposes of the multiple medical images and lets test values of those important test items corresponding to the retrieved test purposes be displayed in the test value display zone.

Preferably, if there are a shared important test item and a not-shared important test item among those for the respective test purposes of the multiple medical images, test values of the shared the important test item are displayed distinguishably from test values of the not-shared the important test item in the test value display zone.

Preferably, if there is a shared the important test item among those for the respective test purposes of the multiple medical images, merely test values of the shared the important test item are displayed in the test value display zone.

Preferably, the medical images are displayed as size-reduced thumbnail images on the test result display screen.

When one of the thumbnail images is selected on the test result display screen, the medical image corresponding to the selected thumbnail image is preferably displayed in an enlarged size that is larger than the thumbnail image.

When the medical image is displayed in the enlarged size, the screen production unit preferably lets test values of the important test items which corresponds to the test purpose be displayed in a manner superimposed on the enlarged medical image.

Preferably, the medical test result display device in accordance with the present invention further comprises a reference value information obtaining section that accesses a reference value storage to retrieve reference value information, the reference value storage storing reference value information that records a reference value range of test values for each of the test items, and a comparing and determining section that compares the reference value range and the test values displayed in the test value display zone, to determine whether the test values are within the reference value range or not, wherein the screen production unit lets test values within the reference value range and test values outside the reference value range distinguishably in the test value display zone.

Preferably, the screen production unit produces a graph on the basis of test values obtained at clinical tests carried out on the patient at different times, the graph indicating temporal changes in test values of the same test item, and lets the graph be displayed in the test value display zone.

Preferably, the screen production unit lets multiple medical images, which have been captured at different times, be displayed in a chronological arrangement on the test result display screen.

A method for operating a medical test result display device in accordance with the present invention, comprises a test result reading step, a test purpose reading step, a screen producing step, an important test item reading step and an image select information accepting step. The test result reading step is reading out medical images and test values of multiple test items from a medical image storage and a test results storage, respectively, wherein the medical image storage stores medical images captured at imaging tests carried out on a patient, each imaging test having at least a test purpose, and the test result storage stores numerical test values that represent test results of multiple test items included in clinical tests carried out on the patient. The test purpose reading step is reading at least a test purpose from a test purpose storage which stores respective test purposes of the imaging tests. The screen producing step is producing a test result display screen having an image display zone in which multiple medical images are capable of being displayed and a test value display zone in which test values of the multiple test items are capable of being displayed. The important test item reading step is reading important test items from an important test item storage according to a test purpose, the important test item storage storing important test items selected for each test purpose from among the multiple test items. The image select information accepting step is accepting image select information that indicates that at least a medical image is selected from among the multiple medical images displayed in the image display zone. Furthermore, the screen producing step comprises the steps of retrieving a test purpose corresponding to the selected medical image on the basis of the image select information, retrieving important test items on the basis of the retrieved test purpose and displaying merely test values of the important test items in the test value display zone on the basis of the retrieved important test items.

According to the present invention, since test values of merely those important test items which correspond to the test purpose of a selected medical image are displayed upon selection of the medical image, it is unnecessary to make cumbersome operations for selecting such important test items that are desired to be displayed. Thereby, the present invention can provide a medical test result display device that makes it easy to check the test values of the important test items according to the test purpose.

### BRIEF DESCRIPTION OF THE DRAWINGS

For more complete understanding of the present invention, and the advantage thereof, reference is now made to the subsequent descriptions taken in conjunction with the accompanying drawings, in which:

FIG. 1 is a schematic diagram illustrating a structure of a medical information system including a test result display device;

FIG. 2 is a schematic diagram illustrating major flows of information in the medical information system;

FIG. 3 is a block diagram illustrating an electrical structure of a computer serving as a test result display device and other devices;

FIG. 4 is a block diagram illustrating a functional structure of test result display device;

FIG. 5 is an explanatory diagram illustrating an example of a medical record display screen;

FIG. 6 is an explanatory diagram illustrating an example of important test item information expressed as a reference table;

FIG. 7 is an explanatory diagram illustrating an example of a test result display screen displaying all test values;

FIG. 8 is an explanatory diagram illustrating an example of a test result display screen displaying only test values of important test items;

FIG. 9 is a conceptual diagram illustrating a sequence of procedures for displaying test values of important test items;

FIG. 10 is an explanatory diagram illustrating a display condition of medical images;

FIG. 11 is a flowchart illustrating a sequence for displaying a test result display screen;

FIG. 12 is an explanatory diagram illustrating an example of a test result display screen in accordance with a second embodiment;

FIG. 13 is a flowchart illustrating a sequence of procedures for displaying the test result display screen in accordance with the second embodiment;

FIG. 14 is an explanatory diagram illustrating another example of a test result display screen in accordance with the second embodiment;

FIG. 15 is an explanatory diagram illustrating an example of important test item information in accordance with a third embodiment;

FIG. 16 is an explanatory diagram illustrating an example of a test result display screen in accordance with the third embodiment; and

FIG. 17 is a flowchart illustrating a sequence of procedures for displaying the test result display screen in accordance with the third embodiment.

### DESCRIPTION OF THE PREFERRED EMBODIMENTS

### First Embodiment

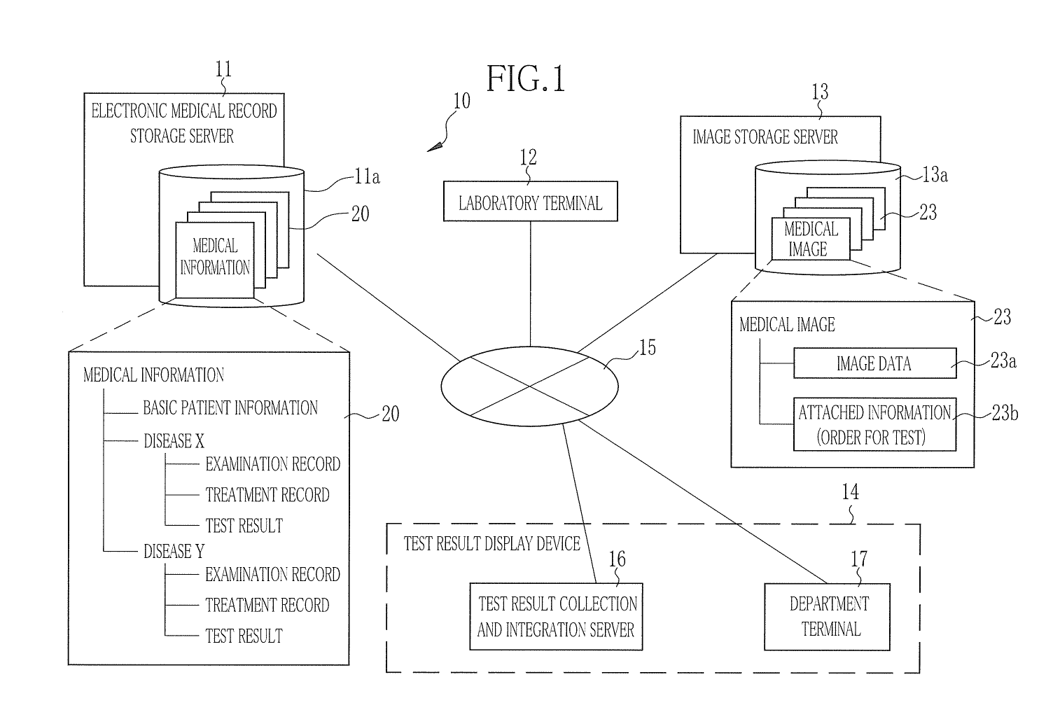

FIG. 1 shows a medical information system 10 that is a computer system serving for management of medical information in a medical facility such as a hospital. The medical information system 10 is constituted of an electronic medical record storage server 11 for storing and managing electronic medical records, a laboratory terminal 12 for inputting results of medical tests, an image storage server 13 for storing medical images, a test result display device (medical test result display device) 14 with a capability of displaying test results, and a network 15, such as LAN (local area network), connecting these components to enable communication therebetween. The test result display device 14 is provided with a test result collection and integration server (hereinafter referred to as the integration server) 16 which collects and integrates test results from the electronic medical record storage server 11 and the image storage server 13 to create a test result display screen, and a department terminal 17 which is installed in each clinical department and is used for browsing the test result display screen.

The electronic medical record storage server 11 is provided with an electronic medical record database (hereinafter referred to as the medical record DB) 11a. The medical record is recording medical information 20 on medical practices for each patient, and the medical information includes patient basic information, records of examinations and treatments carried out on the patient (medical examination records and treatment records), and test results of many kinds of medical tests carried out on the patient. Therefore, the medical record DB 11a corresponds to the test result storage of the present invention. In a case where the patient is affected by multiple illnesses, e.g. disease X and disease Y, the medical examination records, treatment records and test results are recorded as the medical information 20 with respect to the individual diseases X and Y.

The patient basic information includes the name, birth date and sex of the patient, a patient ID, etc. The medical examination records include records of clinical interviews or palpations, etc., doctor findings, attended by injury/disease identifying information that includes an injury/disease name of a suspected or diagnosed injury or disease, and an injury/disease code for identifying the injury or disease.

The treatment records are records of prescribed medicines, operations and treatments carried out on the patient. As test results, results of clinical tests expressed by numerical values, radiographic interpretation findings as results of imaging tests, and link information for the link to a storage location of a medical image (address information on the storage location in the image storage server 13).

The clinical tests include, for example, vital examinations, blood tests, biochemical examinations, urine tests and stool inspections. Each clinical test includes multiple test items. For example, body temperature, blood pressure, pulse, respiratory rate, body weight, etc. may be cited as test items of a vital examination. As test items of a blood test, there may be white blood cell count, red blood cell count, hemoglobin concentration, etc. As test items of a biochemical examination, there may be protein, γ-GTP, etc. In the medical information 20, test values of these test items are recorded as test results along with the date of each clinical test.

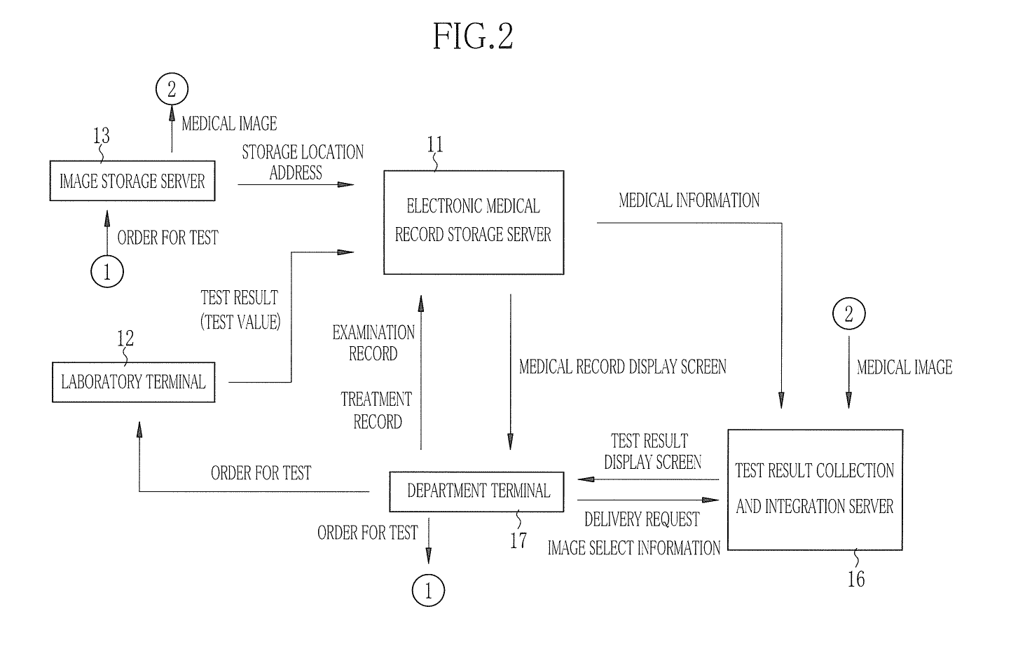

The laboratory terminal 12 is installed in a laboratory for conducting blood tests, vital examinations, etc. As shown in FIG. 2, the laboratory terminal 12 accepts a clinical test to be conducted in the laboratory as it receives a test order for the clinical test from the department terminal 17. In addition, the laboratory terminal 12 is served to input test values of the clinical test in association with the test order at the end of the clinical test. The input test values are sent to the electronic medical record storage server 11. The electronic medical record storage server 11 registers the test values in electronic medical records of the patient who has undergone the clinical test in association with the test order.

The image storage server 13 is provided with an image database (hereinafter referred to as the image DB) 13a that stores medical images 23 captured for imaging tests carried out on patients. The image DB 13a corresponds to the medical image storage of the present invention. There are multiple kinds of medical images 23, such as X-ray images, CT images captured by computer tomography (CT) devices, ultrasonic images and endoscopic images. Each medical image 23 includes image data 23a and attached information 23b attached to the image data 23a. The attached information 23b also contains the test order issued by a doctor when requesting the imaging test, and the test purpose for capturing the medical images 23 is recorded in the test order. Accordingly, the image DB 13a storing the attached information 23b corresponds to the test purpose storage of the present invention. After a medical image 23 is captured, the image storage server 13 stores the captured medical image in the image DB 13a, and transmits memory location address data of this medical image to the electronic medical record storage server 11. The electronic medical record storage server 11 registers the received memory location address data in electronic medical records of the patient who undergone the imaging test.

A test order for a clinical test and that for an imaging test each include multiple items necessary for requesting the test. As items which are included in both test orders, there may be, for example, a patient ID of a patient under the tests, an ID of a doctor who requests the tests, the contents of the test, the purpose of the test, the date of the test, etc. As the test purpose is recorded an injury/disease name and a concrete purpose of the test, e.g. for determining the injury or disease of the patient, or for following-up the injury or disease that has been determined. Note that, to say nothing of a follow-up test, an injury/disease name is often recorded even in the test purpose for determining the injury or disease, because a doctor previously estimate the injury or disease that can affect the patient, and orders a test for determining whether the patient is affected by the estimated injury or disease or not. That is, the test purpose clearly indicates the name of a designated injury or disease, e.g. the purpose is for diagnosing lung cancer, pneumonia, stomach cancer, etc.

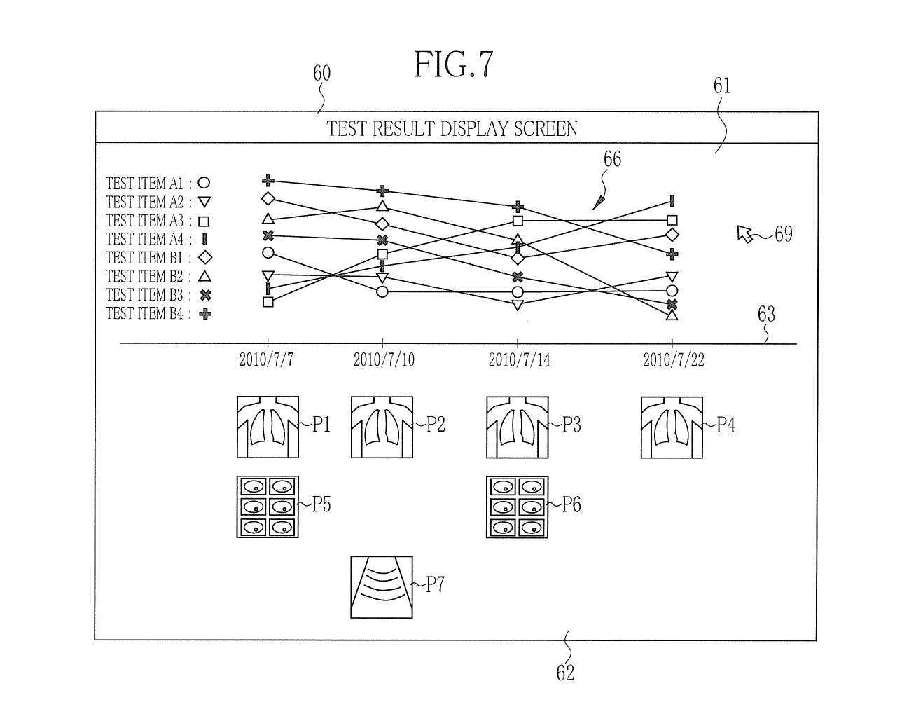

The integration server 16 has a capability of accessing the electronic medical record storage server 11 and the image storage server 13 to collect the medical information 20 including test values and the medical images 23, and produces a test result display screen 60 (refer to FIG. 7) that integrates the test values included in the medical information 20 and the medical images 23. The integration server 16 produces the test result display screen 60 upon receipt of a browse request, and delivers the produced test result display screen 60 to the department terminal 17.

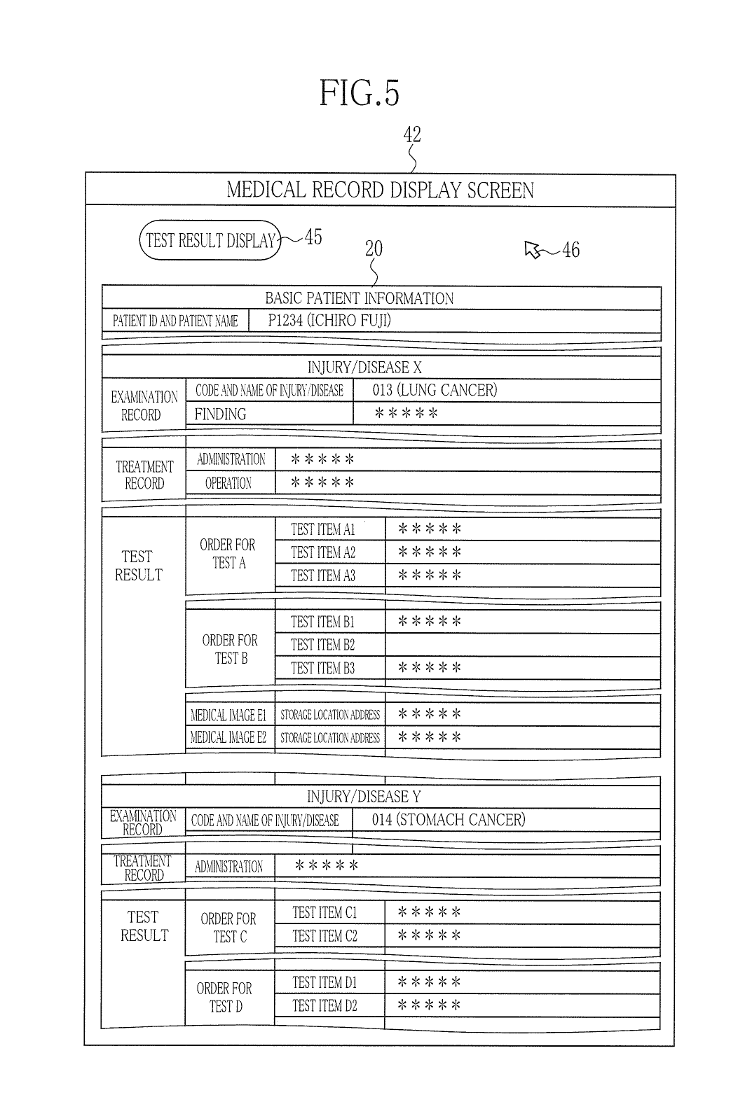

The department terminal 17 is a terminal installed in individual clinical departments, including those for internal medicine, surgery, otolaryngology and ophthalmology, and functions as a display terminal for displaying the test result display screen 60 received from the integration server 16. The department terminal 17 also serves as a medical record terminal that accesses the electronic medical record storage server 11 to input examination records and treatment records therein or to receive a medical record display screen 42 (refer to FIG. 5) from the electronic medical record storage server 11, to display the medical information 20 for browsing.

The electronic medical record storage server 11, the laboratory terminal 12, the image storage server 13, the integration server 16 and the department terminal 17 are each configured on the basis of a computer, such as a personal computer, a server computer or a workstation, by installing a control program, such as an operating system, and an application program, such as a client program or a server program, therein.

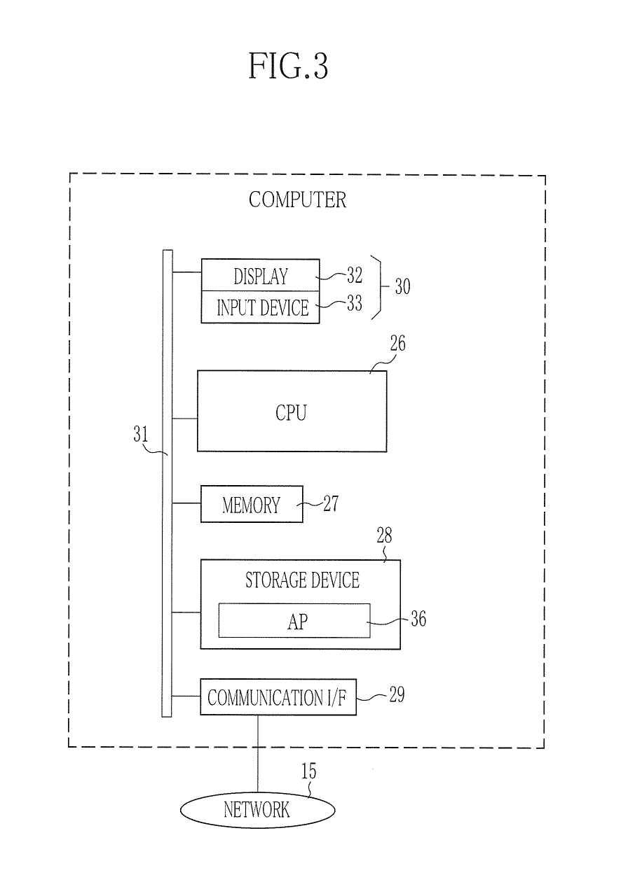

As shown in FIG. 3, one computer is provided with a CPU 26, a memory 27, a storage device 28, a communication interface (UF) 29 and a console 30. These components are interconnected through a data bus 31. The console 30 includes a display 32 and input devices 33, such as a keyboard and a mouse.

The storage device 28 is for example a hard disk drive (HDD) that stores the control program and the application program (AP) 36. In a computer for establishing a DB, a storage device 28 for the DB, for example, a disk array having an array of HDDs, is provided in addition to the HDD for storing the programs. The disk array may be integrated in the main body of the computer, or may be provided separately from the main body and connected to the main body through a cable or network.

The memory 27 is a work memory for the CPU 26 to execute processing. The CPU 26 loads the control program from the storage device 28 to the memory 27 and executes processing according to the program, controlling the components of the computer comprehensively. The communication I/F 29 is a network interface for controlling data transmission over the network 15.

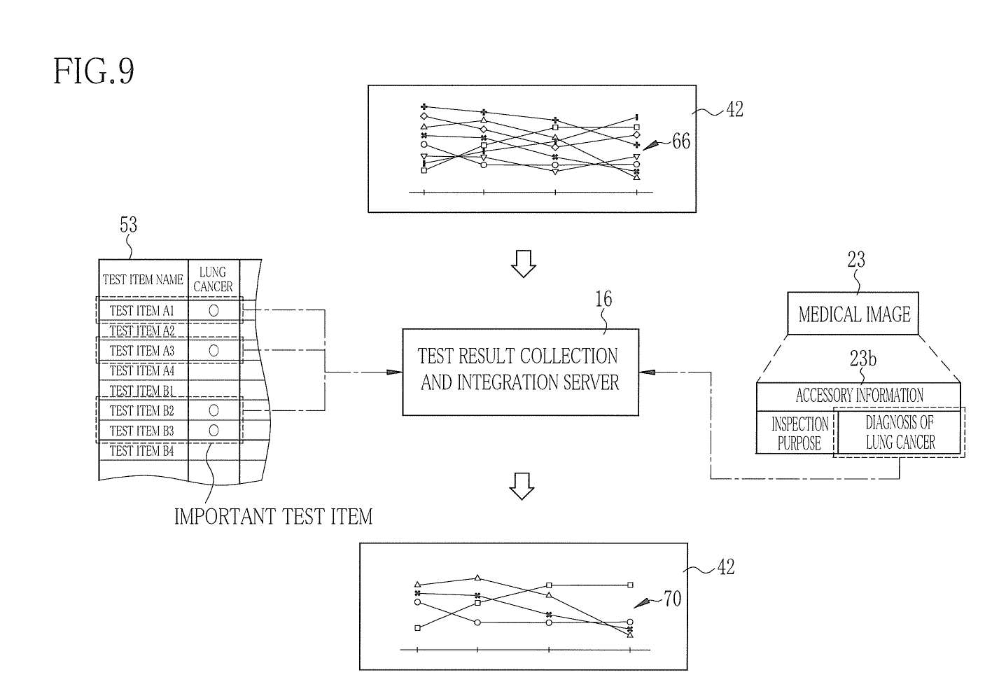

The department terminal 17 is installed with client programs as the AP 36, such as software for medical records, enabling accessing the electronic medical record storage server 11 to browse or edit a medical record display screen 42 that displays the medical information 20, and viewer software enabling accessing the integration server 16 or the image storage server 13 to browse a test result display screen 60 (refer to FIG. 9) or images. The client program is a browser that receives screen data described in a markup language, e.g. extensible markup language (XML), and displays the screen on the display 32. The screen data includes operation screens based on graphical user interface (GUI).

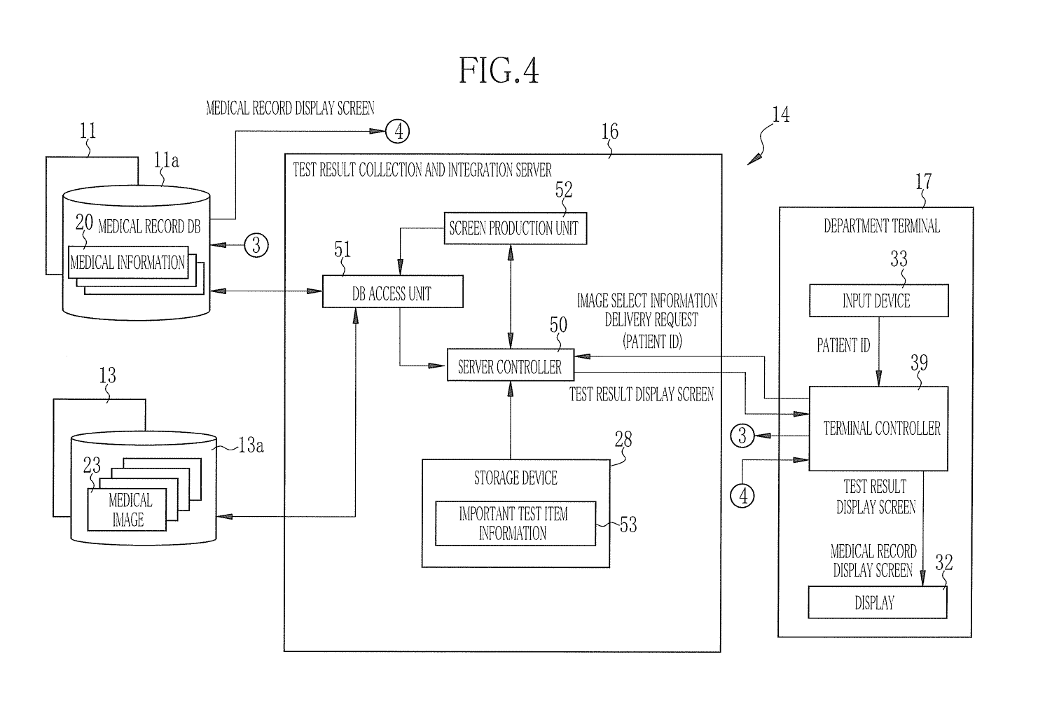

As shown in FIG. 4, the CPU 26 of the department terminal 17 functions as a terminal controller 39 when the client program is executed. The terminal controller 39 accepts an input of information and various operation commands through the input device 33. When a patient ID is designated and a display command for displaying a medical record display screen 42 is received on the terminal controller 39, the terminal controller 39 sends a delivery request including the designated patient ID to the electronic medical record storage server 11 and receives the medical record display screen 42 corresponding to the designated patient ID. The terminal controller 39 controls displaying the received medical record display screen 42 on the display 32.

As shown in FIG. 5, the medical record display screen 42 is provided with display columns for respectively displaying patient basic information, medical examination records, treatment records and test results included in the medical information 20. In addition, in a case of the medical information 20 that relates to a patient who is affected by multiple illnesses, e.g. diseases X and Y, the columns for medical examination records, the treatment records and test results are displayed individually for each illness. On the medical record display screen 42, the medical examination record column displays the name of disease or injury and a corresponding injury/disease code as injury/disease identifying information and the findings of the doctor, etc. The treatment record column displays information on administration, operation and treatment. As the information on administration, the names of medicines, the dose amounts of the medicines, the duration of the administration, etc. are recorded. As the information on operation, the operation name, the operation date, the surgeon, etc. are recorded.

In the test result column, test results of a clinical test and information on the link to a storage location of medical images captured for an imaging test, e.g. medical images E1 and E2, are displayed (the link information being information of storage location address in the image storage server 13). The test results are classified according to the test orders, e.g. test orders A, B, C and D, and test items and test values are displayed for each test order. For example, the test items include test items A1, A2, A3, B1, B2, B3, C1, C2, D1 and D2, and the test values of the respective test items are displayed. As test items and test values for a clinical test, for example for a vital examination, test items, such as body temperature, blood pressure and blood oxygen saturation, and test values of these test items are displayed. For a blood test, test items, such as red blood cell count, white blood cell count and hemoglobin concentration, and test values of these test items are displayed. Furthermore, for a biochemical examination, test items, including C-reactive protein, and test values thereof are displayed.

The medical record display screen 42 is provided with a test result display button 45 above the display columns for the medical information 20. The test result display button 45 is an operation button for instructing to display the test result display screen 60 that displays the results of imaging tests and clinical tests carried out on the patient in an integrated or comprehensive manner. When a mouse pointer 46 is operated to click on the test result display button 45 through the input device 33, a display command for the test result display screen 60 enters the terminal controller 39.

As shown in FIG. 4, the terminal controller 39 reads out, upon receipt of the display command for the test result display screen 60, a patient ID from the medical information 20 displayed on the medical record display screen 42. In addition, the terminal controller 39 sends a delivery request including the read patient ID to the integration server 16. Upon receipt of the test result display screen 60 in correspondence with the delivery request, the terminal controller 39 controls displaying the test result display screen 60 on the display 32.

The integration server 16 is installed with a server program as an AP 36 for executing processing in accordance with the request from the client. The AP 36 corresponds to the medical test result display program of the present invention. The CPU of the integration server 16 executes the server program to function as a server controller 50, a DB access unit 51, a screen production unit 52. The server controller 50 is connected to the storage device 28. The storage device 28 stores important test item information 53, and corresponds to the important test item storage of the present invention.

The server controller 50 receives the delivery request form the department terminal 17 through the communication I/F 29, and controls the components of the integration server 16. The DB access unit 51 corresponds to the test result reading section of the present invention, and accesses the medical record DB 11a and the case DB 15a through the communication I/F 29, to read out such medical information 20 and medical images 23 that correspond to the patient ID included in the delivery request. The screen production unit 52 produces a test result display screen 60 on the basis of the medical information 20 and the medical images 23.

The screen production unit 52 reads out all test values of the test items of a clinical test from the medical information 20 that is retrieved by the DB access unit 51. For example, as shown in FIG. 5, if the medical information 20 contains records of test results relating to multiple diseases X and Y, all test values of the test items A1 to A3, B1 to B3, C1, C2, D1 and D2 of these diseases X and Y are read out. The screen production unit 52 also produces thumbnail images of all medical images 23 retrieved by the DB access unit 51. The thumbnail images are produced by reducing the size of image data 23a of the medical images 23. It may be possible to previously produce and store the thumbnail images in the image DB 13a in addition to the image data 23a, to read out the thumbnail images. The screen production unit 52 creates a test result display screen 60 on the basis of the read test values and the produced thumbnail images. The produced test result display screen 60 is delivered to the department terminal 17 through the server controller 50 and the communication I/F 29.

The server controller 50 also functions as the image select information receiving section of the present invention, and accepts image select information including identification information of the thumbnail image selected on the department terminal 17 by means of the test result display screen 60 and the input device 33. The screen production unit 52 also function as the test purpose reading section of the present invention, which refers to the attached information 23b of the medical image 23 that corresponds to the selected thumbnail images, and reads out the test purpose from the test order included in the attached information 23b, thereby to determine the injury/disease name by the contents of the test purpose. For example, if the test purpose is “diagnosis of lung cancer”, the server controller 50 determines that “lung cancer” is the name of disease. The server controller 50 also obtains an injury/disease code corresponding to the determined injury/disease name.

The screen production unit 52 functions also as an important test item reading section, which accesses the storage device 28 to refer to the important test item information 53 and obtains those important test items corresponding to the injury/disease code determined on the basis of the purpose of the test. The screen production unit 52 then revises the test result display screen 60.

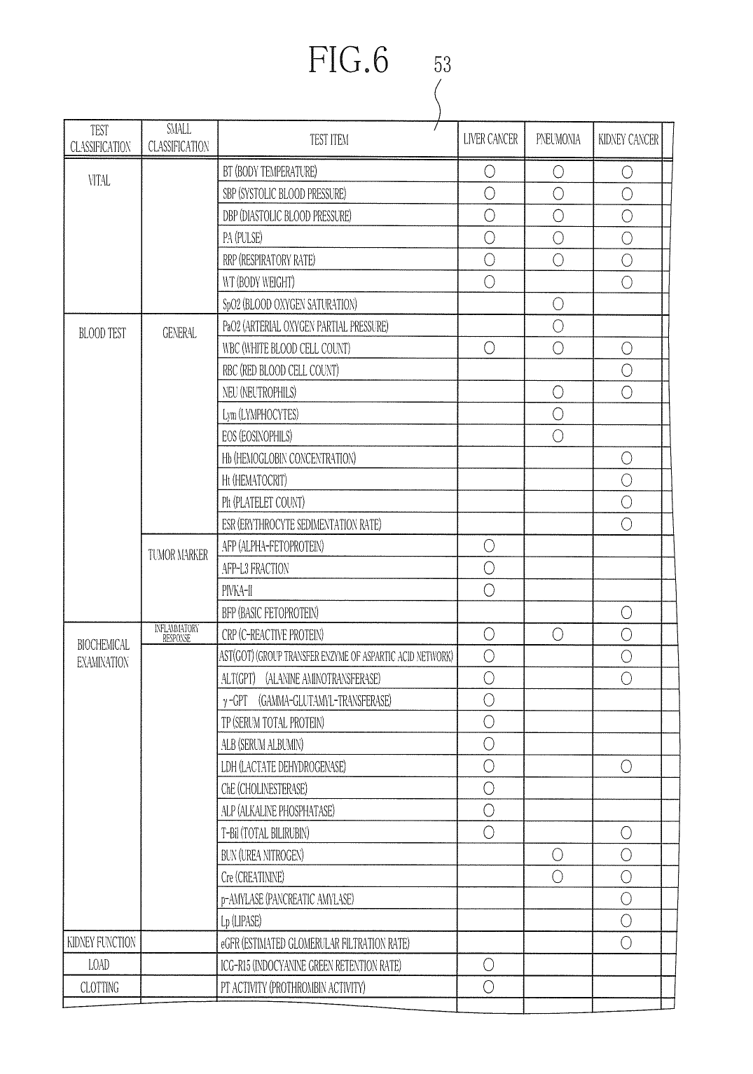

As shown in FIG. 6, the important test item information 53 records important test items for each kind of injuries and diseases among a lot of test items included in clinical tests. The important test items are such test items that should be referred to as particularly important test items during the medical practice. The important test items are selected from among a lot of test items, and set up for each injury or disease individually. For example, the important test item information 53 is largely categorized by the kinds of clinical tests, such as vital examination, blood test and biochemical examination, and test items involved in the individual kinds of clinical tests are arranged in the subdivisions of each category. The names of injuries and diseases, such as lever cancer, pneumonia and kidney cancer, are arranged in a row. Although only the injury/disease names are shown in FIG. 6, the injury/disease codes are also recorded in the important test item information 53 in addition to the injury/disease names.

In FIG. 6, marks “∘” indicate important items for individual kinds of injuries and diseases. As important items for “pneumonia”, items “SpO2”, “Lym”, “EOS”, “CRP”, etc. are marked with “∘”. The important items vary depending on the kinds of injuries and diseases. For instance, among test items of the blood test, the item “red blood cell count (RBC)” is not an important item for “pneumonia”, but is regarded as an important item for “kidney cancer”. The important test item information 53 is stored in the form of a reference table in the storage device 28, which records important test items for the respective injuries and diseases in association with the individual injuries and diseases. Since an injury or disease is indicated in the test purpose, it is possible to read out those test items important for the test purpose with reference to the important test item information 53 that records important test items in association with each injury or disease.

Note that the important test item information 53 shown in FIG. 6 is just an example, wherein “lever cancer”, “pneumonia” and “kidney cancer” are recorded as the diseases, but in practice, “pneumonia” for instance is classified more finely according to the responsible microorganisms: “pneumococcal pneumonia”, “klebsiella pneumoniae pneumonia”, “influenza bacillus pneumonia”, “pseudomonas aeruginosa pneumonia”, “legionella pneumonia”, etc. In the important test item information 53, important test items are recorded for each of the fine-classified injuries and diseases.

The important test item information 53 is provided, for example, as a content of the application program for the integration server 16, but may be newly created or updated in the medical facility. For this purpose, initially setup important items can be edited by the user so as to add or delete some items as needed. Since the decision on which test items are regarded as important among a plurality of test items can differ from doctor to doctor, the capability of editing the important test items enables the individual doctors to customize the important test items. The creation and revision of the important test item information 53 may be made, for example, on the console of the integration server 16 or the department terminal 17.

FIG. 7 shows the test result display screen 60 produced in the screen production unit 52 on the basis of the medical information 20 and the medical images 23, which are read by the DB access unit 51. The test result display screen 60 is provided with a test value display zone 61 located on the upper side of the screen and an image display zone 62 located below the test value display zone 61. A time axis 63 for representing the time flow is displayed in between the test value display zone 61 and the image display zone 62, the time flowing to the right on the time axis in this example. The times when the clinical tests and the imaging tests were carried out are plotted on the time axis 63, and the dates thereof are displayed below.

The test value display zone 61 displays a test value graph 66 which shows temporal changes in test values of all test items and is created on the basis of test values of multiple medical tests carried out a number of times on a patient, which are read out from the medical information 20. On the test value graph 66, the horizontal axis represents the time, and the vertical axis represents the magnitude of the test values, and the time represented by the horizontal axis is set up according to the time axis 63. In the present example, clinical tests were conducted four times on 7^{th}, 10^{th}, 14^{th} and 22^{nd} of July 2010, respectively, and the test value graph 66 is created for each test item. The test value graph 66 is a time-series graph that plots the test values of the test items A1 to A4 and B1 to B4 of the clinical tests as plot points arranged in a time series fashion and interconnects the plot points of each item with a straight line. Since the test values of the respective test items A1 to A4 and B1 to B4 are expressed in different units, the magnitudes of the test values expressed along the vertical axis are standardized such that relative changes in the test values can be seen with respect to each test item.

Note that captions indicating the test items and the numerical test values may be displayed beside the plotted markers. Furthermore, in order to discriminate the individual test items, the plot points have different shapes from item to item in the illustrated example, but it is possible to use different colors or sizes of plot points, different colors, types or boldness of the lines in the graph.

In the image display zone 62, thumbnail images P1 to P7 are arranged along the time axis 63 according to the respective dates of capture. The vertical positions of the thumbnail images P1 to P7 are determined according to the kinds of imaging tests. For example, the thumbnail images P1 to P4 arranged on the uppermost line are X-ray images acquired by an X-ray radiography, and the thumbnail images P5 and P6 on the second line are CT images acquired by a computed tomography scanning, whereas the thumbnail image P7 on the bottom line is an ultrasonic image acquired by an ultrasonic inspection. If there are other kinds of medical images, such as endoscopic images acquired by an endoscopy, thumbnail images thereof may be displayed, for example, below the thumbnail image P7.

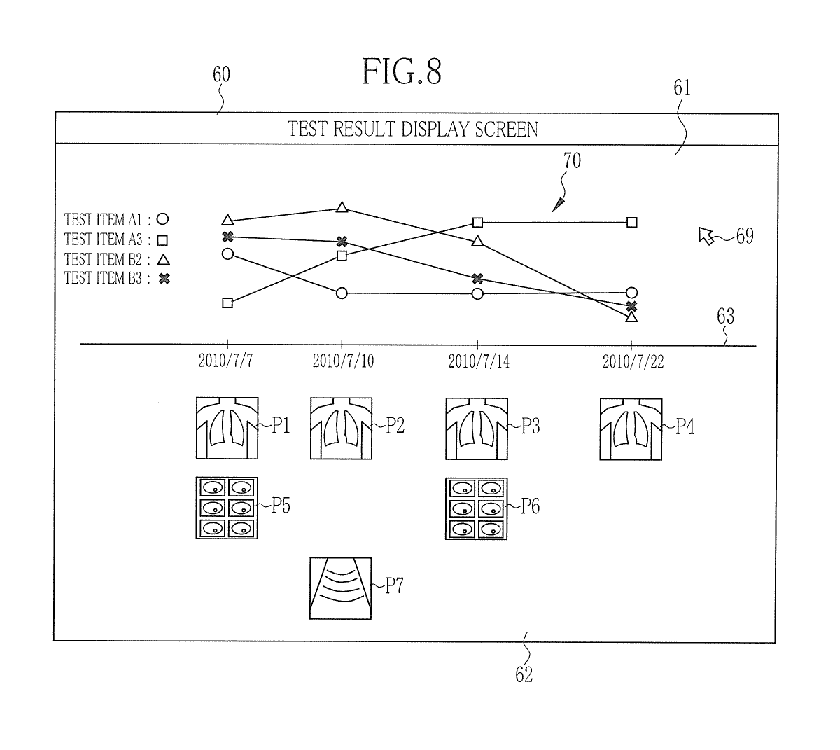

When the mouse pointer 69 is operated through the input device 33 to select any of the thumbnail images P1 to P7 on the test result display screen 60 shown in FIG. 7, the screen production unit 52 revises the test result display screen 60. FIG. 8 shows a test result display screen as revised by the screen production unit 52, which displays a test value graph 70 showing only those important test items corresponding to the test purpose of the selected thumbnail image, and test values of other test items are not displayed. In the illustrated example, only test values of test items A1, A3, B2 and B3 are displayed in the graph 70. Accordingly, the test result display screen 60 after the selection of a thumbnail image is improved in visibility of test values of important test items.

FIG. 9 conceptually shows a sequence of processing in the integration server 16 from the selection of a thumbnail image to the revision to the test value graph 70 including only important test items. The processing sequence in the integration server 16 includes a step of retrieving the test purpose of the medical image 23 on the basis of the selection of the thumbnail image, a step of retrieving important test items on the basis of the test purpose, and a step of revising the display contents in the test value display zone 61 of the test result display screen 60, changing from the test value graph 66 relating to all test values to the test value graph 70 showing only test values of the important test items.

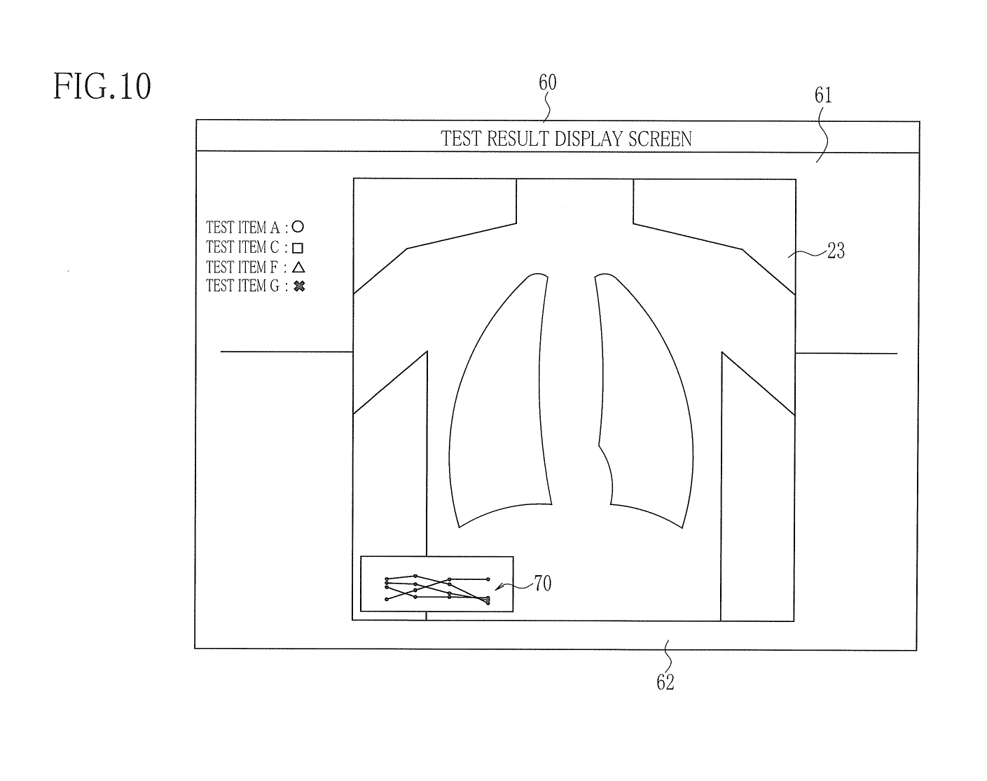

Furthermore, as shown in FIG. 10, when any of the thumbnail images P1 to P7 is double-clicked on, the screen production unit 52 displays the double-clicked medical image 23 in a larger size than the thumbnail images on the test result display screen 60. In that case, the contents in the test value display zone 61, i.e. the test value graph 70 is displayed superimposed on the enlarged medical image. Thus, the test values can be observed simultaneously with the enlarged medical image 23. Note that the test value graph 70 can be moved on the medical image 23 to facilitate inspecting an affected site in the medical image 23.

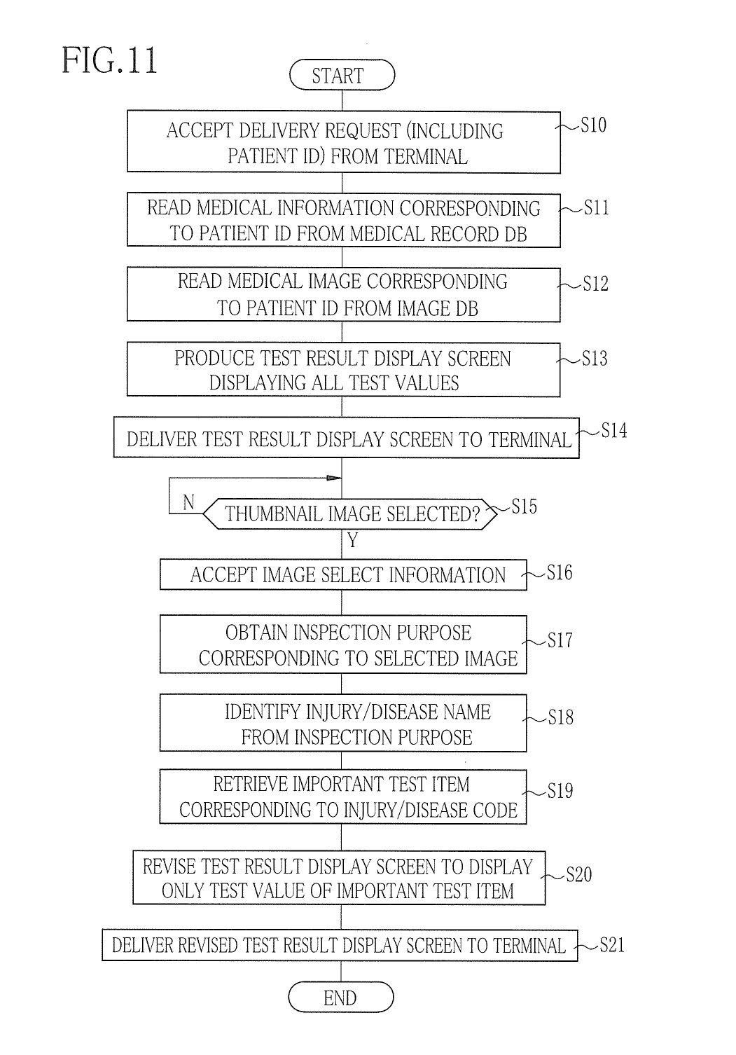

The operation for displaying a test result display screen 60 by the above configuration will be described with reference to the flowchart shown in FIG. 11. When the terminal controller 39 accepts an input of a display command for the test result display screen 60 on the medical record display screen 42, the terminal controller 39 reads out the patient ID from the medical information 20 displayed on the medical record display screen 42, and sends a delivery request, including the read patient ID, to the integration server 16. The integration server 16 receives the delivery request from the department terminal 17 (S10).

The DB access unit 51 accesses the medical record DB 11a through the communication I/F 29, to read out the medical information 20 corresponding to the patient ID included in the delivery request (S11). In addition, the DB access unit 51 accesses the image DB 13a through the communication I/F 29, to read out medical images 23 corresponding to the patient ID included in the delivery request (S12).

The screen production unit 52 produces a test result display screen 60 showing all test values relating to the patient on the basis of the medical information 20 and the medical images 23, which are read by the DB access unit 51 (S13). Specifically, the screen production unit 52 reads out test values of all test items of medical tests carried out a number of times on the patient from the medical information 20, to create the test value graph 66 showing temporal changes in test values of the respective test items. In addition, the screen production unit 52 produces thumbnail images P1 to P7 of all medical images 23. The screen production unit 52 produces the test result display screen 60 on the basis of the test value graph 66 and the thumbnail images P1 to P7. The produced test result display screen 60 is delivered to the department terminal 17 through the server controller 50 and the communication I/F 29 (S14).

When the terminal controller 39 receives the test result display screen 60 in correspondence with the delivery request, the terminal controller 39 controls displaying the test result display screen 60 on the display 32. Thus, the test result display screen 60 displays the test value graph 66 showing temporal changes in the test values of all test items read out from the medical information 20, and the thumbnail images P1 to P7 of all medical images 23 captured from the patient.

When a thumbnail image is selected on the test result display screen 60 through the input device 33 (YES in S15), the server controller 50 accepts the image select information from the department terminal 17, including identification information of the selected thumbnail image (S16). The screen production unit 52 refers to the attached information 23b of the medical image 23 corresponding to the selected thumbnail image, to retrieve the test purpose from the test order included in the attached information 23b (S17). Then, the screen production unit 52 determines the injury/disease name by the contents of the retrieved test purpose (S18). Furthermore, the screen production unit 52 retrieves the injury/disease code corresponding to the determined injury/disease name from the medical information 20. That is, the medical information 20 includes a plurality of injury/disease names and injury/disease codes, and the screen production unit 52 obtains the injury/disease code corresponding to the determined injury/disease name from among the plurality of injury/disease codes included in the medical information 20.

The screen production unit 52 accesses the storage device 28 to refer to the important test item information 53, thereby to retrieve important test items corresponding to the obtained injury/disease code (S19). Then, the screen production unit 52 revises the test result display screen 60 so as to display only test values of the important test items in the test value display zone 61 (S20). Specifically, the test result display screen 60 is revised such that a test value graph 70 showing only the test values of the obtained important test items is displayed in the test value display zone 61. The revised test result display screen 60 is delivered to the department terminal 17 through the server controller 50 and the communication 1/F 29 (S21).

The terminal controller 39 controls displaying the revised test result display screen 60 on the display device 32. Thus, the test value graph 70 which shows only the test values of those important test items which correspond to the test purpose of the selected thumbnail image is displayed in the test value display zone 61 of the test result display screen 60.

The present embodiment makes it possible to display only test values of important test items corresponding to the test purpose of a thumbnail image (medical image) that is selected from among thumbnail images of a plurality of medical images captured for medical tests. Therefore, it is unnecessary to select important test items one by one to display test values of these items, improving operability. Furthermore, since only test results of important test items are displayed and unnecessary information is omitted from the screen, the visibility of test values of the important test items is improved. This may prevent misdiagnosis due to misreading of the test values. In addition, because the test results is displayed as a test value graph showing temporal changes in the test values, the present invention is very useful for deciding on therapeutic strategies or evaluating therapeutic effects.

The above embodiment has been described with respect to a case where a single thumbnail image is selected on the test result display screen 60, but it may be possible to select more than one thumbnail images. In that case, the screen production unit 52 retrieves the test purpose and important test items and display test values of the retrieved important test items in the test value display zone 61 with respect to each of the selected thumbnail images. This makes it possible to browse the test values corresponding to multiple test purposes at the same time, which is effective for medical practices in a case where multiple illnesses might intercurrently occur.

In the above embodiment, the test purpose is recorded in the attached information 23b, and the image DB 13a functions as the test purpose storage, but the test purpose may be recorded separately from the attached information 23b. Also in this case, it is necessary to record the test purpose in association with the medical image 23. If only the medical images 23 are associated with the respective test purposes, it is possible to constitute a test purpose storage separately from the image DB 13a.

### Second Embodiment

Next, a second embodiment of the present invention will be described. Note that, in the present embodiment, the same components will be designated by the same reference numerals as in the first embodiment, so that the detailed description thereof will be omitted. In addition to the configuration of the first embodiment, the second embodiment is configured to have a capability of displaying shared important test items distinguishably from not-shared important test items among those important test items corresponding to the respective test purposes of a plurality of medical images 23, of which thumbnail images are selected on the test result display screen 60. As indicated in the first embodiment, individual medical images 23 have respective test purposes which are recorded in individual attached information 23b, and there are important test items assigned to each test purpose individually.

Since the important test items are recorded for each individual test purpose, if a plurality of medical images are captured for the same test purpose, all the important test items, corresponding to the same test purpose, are shared among these medical images. However, if one medical image has one test purpose and another medical image has another test purpose, there may be some important test items which correspond to both test purposes, and other important test items may be assigned only to one test purpose or the other test purpose. In that case, it may be preferable if it is possible to distinguish the important test items shared among those corresponding to the different test purposes from the not-shared test items when observing the test results.

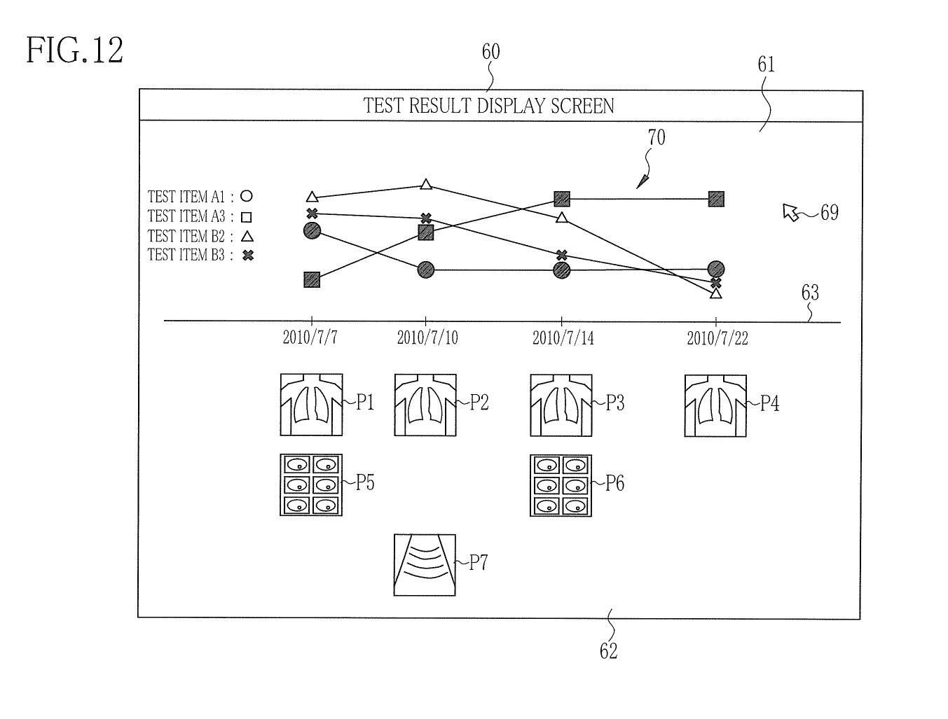

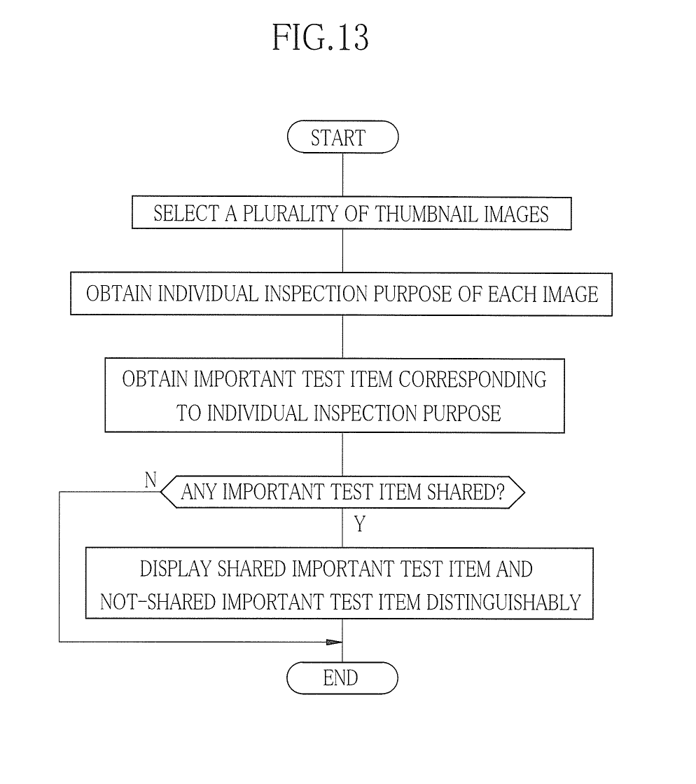

Referring to FIGS. 12 and 13, if for instance two thumbnail images P3 and P6 are selected, the screen production unit 52 retrieves respective test purposes of these images P3 and P6, and then retrieves important test items assigned to the test purposes respectively. If the test purposes of the images P3 and P6 are different, the screen production unit 52 compares the important test items assigned to the test purpose of the image P3 with those assigned to the test purpose of the image P6, to determine whether there are such important test items that are common to both test purposes. For example, assuming that test items

A1 and A3 are common important test items to the test purposes of the images P3 and P6 and that test items B2 and B3 are assigned to either one of the test purposes of the images P3 and P6, the screen production unit 52 determines the test items A1 and A3 to be the shared important test items, and the test items B2 and B3 to be the not-shared important test items.

Then, as shown in FIG. 12, the screen production unit 52 displays test values of the test items A1 and A3, which are determined to be the shared important test items, and test values of the test items B2 and B3, which are determined to be the not-shared important test items, distinguishably on the test result display screen 60. For example, the screen production unit 52 makes the plot points indicating the test values of the shared important test items A1 and A3 greater than the plot points indicating the test values of the not-shared important test items B2 and B3, for the sake of distinguishability. In addition, as for the test items A1 and A3, the plot points are displayed as filled marks while the plot points are usually displayed as outline marks for these items. As a method of making the shared important test items distinguishable, it may be possible to display the plot points in a different color or in a blinking fashion. It may also be possible to interconnect the plot points with a bolder line or a different-colored line for the shared important test items. Furthermore, because the shared important test items should be distinguishable, it is possible to make the not-shared important test items inconspicuous, for example, by displaying the test values thereof fainter than those of the shared important test items. Thus, the shared important test items become relatively conspicuous and more visible.

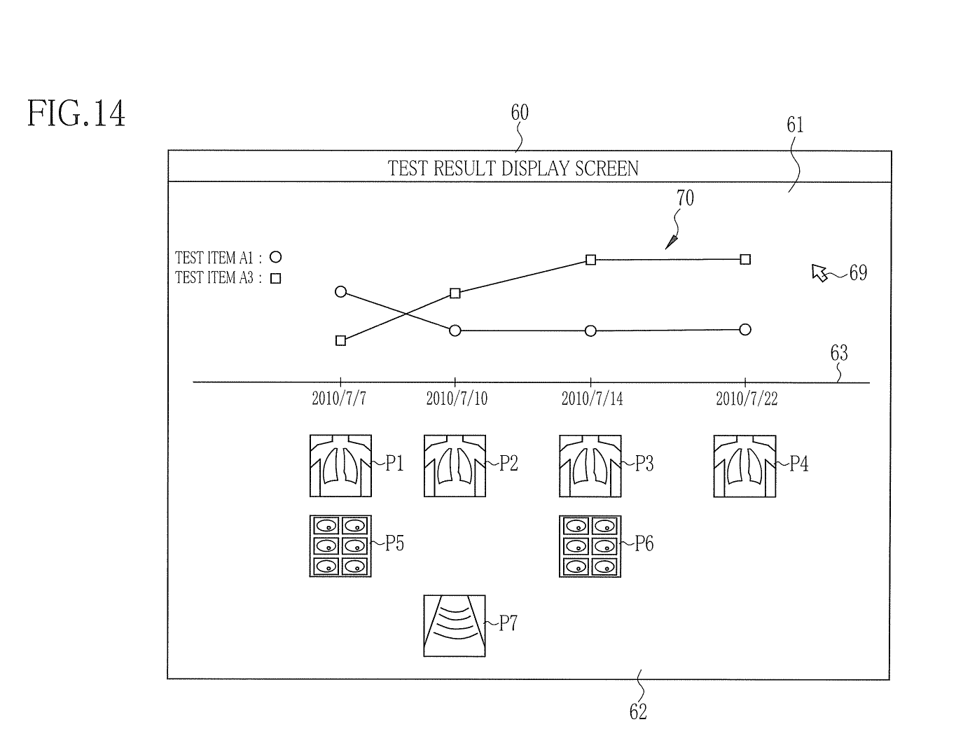

Instead of displaying the shared important test items and the not-shared important test items distinguishably, it is possible to display only the shared important test items and not to display the not-shared important test items. For example, as shown in FIG. 14, in the case where the test items A1 and A3 are the shared important test items, the screen production unit 52 displays merely the shared test items A1 and A3 in the test value display zone 61.

### Third Embodiment

Next, a third embodiment of the present invention will be described. The third embodiment is configured to have a capability of accessing a reference value storage, which stores reference value information recording a reference value range of test values for each test item, to retrieve the reference value information and compare the reference value information with test values displayed in the test value display zone, thereby to display test values within the reference value range and those outlying the reference value range distinguishably. Note that other features of the third embodiment are equivalent to those of the first embodiment, and the same components will be designated by the same reference numerals as in the first embodiment, so that the detailed description thereof will be omitted.

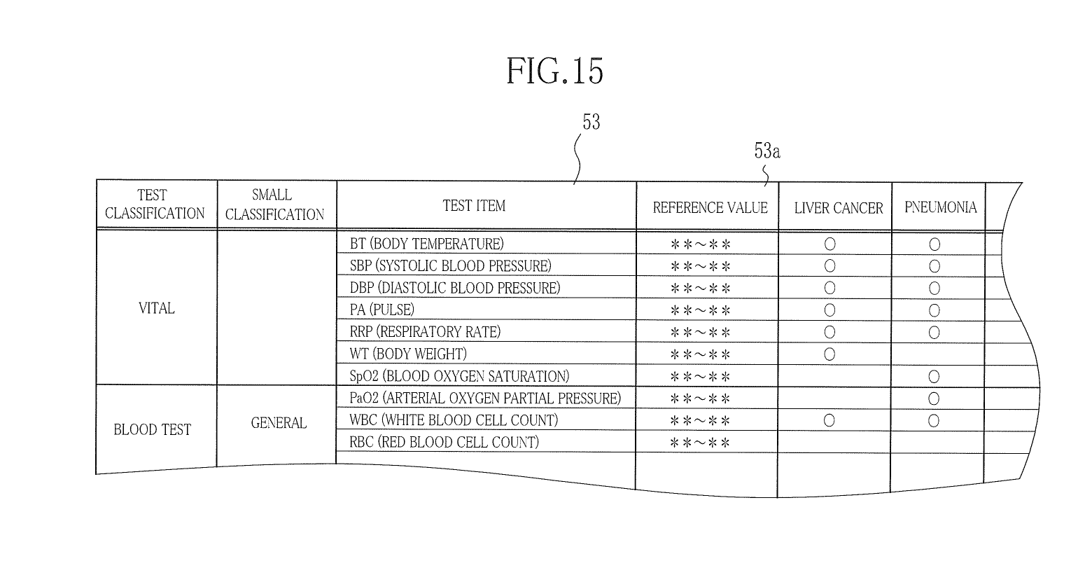

According to the present embodiment, as shown in FIG. 15, reference value information 53a indicating the reference value ranges is added to important test item information 53, so that the storage device 28 doubles as the reference value information storage. It is of course possible to provide the reference value information storage separately from the important test item storage.

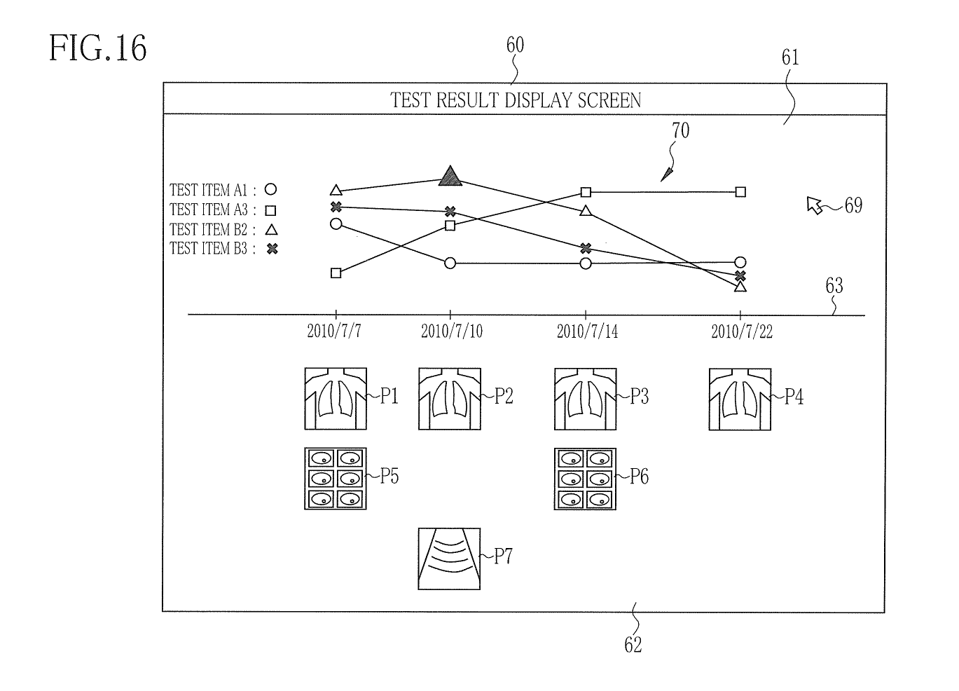

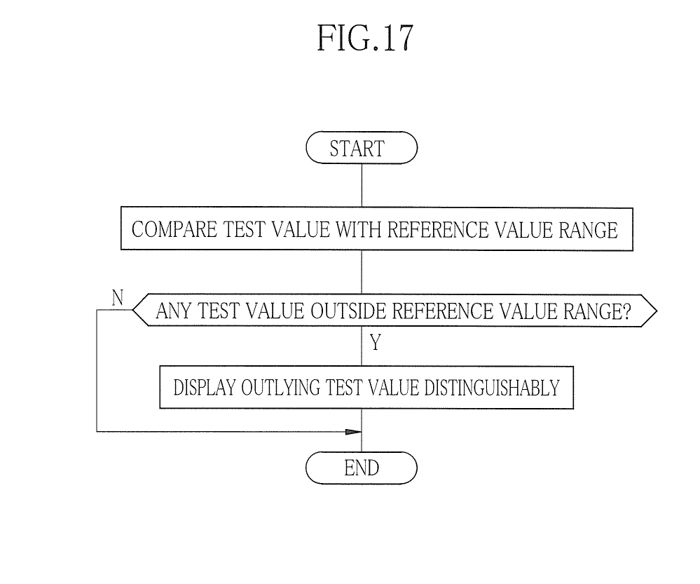

As shown in FIGS. 16 and 17, the screen production unit 52 also functions as a reference value information obtaining section and a comparing and determining section of the present invention, and obtains the reference value information 53a as well as the important test items from the important test item information 53. Then, the screen production unit 52 compares the reference value range with test values displayed in the test value display zone 61 to determine whether the test values are within the reference value range or not. If there is an outlying test value that is excluded from the reference range, the screen production unit 52 displays the plot point of the outlying test value distinguishably from those of the test values within the reference range, for example, by enlarging the plot point of the outlying test value. In addition, the plot point of the outlying test value is expressed by a filled mark, while ordinary plot points are expressed by outline marks.

As a method of making the outlying test value distinguishable, it may be possible to display the plot point thereof in a different color or in a blinking fashion. Because the outlying test value should be distinguishable, it is possible to make the test values within the reference value range inconspicuous, for example, by displaying the test values within the reference value range fainter than the outlying test value. It may also be possible to display only outlying test values. Note that the reference value information 53a is incorporated in the important test item information 53 in the present embodiment, but it is possible to provide and store the reference value information separately from the important test item information 53 in the storage device 28.

Any of the above embodiments have been described with reference to examples wherein the test purpose is determined by the injury or disease. However, there may be cases wherein different test purposes are assigned to the same injury or disease. For example, imaging tests for tumors in kidney include an imaging test for diagnosis on whether the tumors are benign or malignant and an imaging test for follow-up observation of the tumor after the tumor was diagnosed as a malignant kidney cancer. In that case, the imaging tests before and after the diagnosis have different test purposes even while the same injury or disease is assigned thereto: the test purpose of the imaging test before the diagnosis is to determine whether the tumors are benign or malignant, whereas the test purpose of the imaging test after the diagnosis is to follow up the tumor. The important test items for the benign/malignant determination may different from those for the follow-up. Therefore, if the test purposes include other factors, such as benign/malignant determination and follow-up observation, than diagnosis of injuries and diseases, the test purposes may be classified according to these factors in addition to the classification according to injuries and diseases. Then, a set of important test items for each test purpose classified this way are set up in the important test item information 53.

Furthermore, there are such imaging tests that are conducted before the diagnosis without any prediction of injury or disease. In that case, it is possible to designate the test purpose by the name of an internal organ in place of the injury/disease name, for example, a fundamental check-up of lung or a fundamental check-up of kidney. Then important test items corresponding to a test purpose designated by an internal organ are set up in the important test item information 53. Thus, the test purposes may be designated by other factors than the injury or disease. In general, however, an injury or disease is designated as the test purpose, and it is considered to be hard to select important test items before determining the injury or disease. Therefore, it is preferable to designate an injury or disease as the test purpose.

Note that the test result display screen displays test values in the form of a test value graph in each of the above embodiments, but it is possible display the test values as they are. In that case, it is preferable to display an outlying test value in such a manner as to indicate that this value is outside a reference value range. In addition, if a test value of the same item as a displayed test value, which was detected by a test carried out on the same patient at a different time, is out of the reference value range, it is possible to indicate that fact.

Although the test result display device 14 is constituted of the integration server 16 and the department terminal 17, it is possible to provide a department terminal 17 with the function of the integration server 16 to embody a test result display device only with the department terminal 17.

Furthermore, the above embodiments have been described with respect to those examples which embody the test result display device using the department terminal 17 that serves also as a medical record terminal, but it is possible to embody the test result display device using a specific terminal that is provided with a test result display function only and does not function as a medical record terminal.

Although the present invention has been fully described by the way of the preferred embodiments thereof with reference to the accompanying drawings, various changes and modifications will be apparent to those skilled in the art. Therefore, unless these changes and modifications depart from the scope of the present invention, they should be construed as included therein.

What is claimed is:

1. A medical test result display device, comprising:
a test results reading section which accesses a medical image storage and a test results storage to read out medical images and test values of multiple test items, wherein the medical image storage stores medical images captured at imaging tests carried out on a patient, each imaging test having at least a test purpose, and the test result storage stores numerical test values that represent test results of multiple test items included in clinical tests carried out on the patient;
a test purpose reading section that accesses a test purpose storage to read out at least a test purpose, the test purpose storage storing respective test purposes of the imaging tests;
a screen production unit that produces a test result display screen having an image display zone in which multiple medical images are capable of being displayed and a test value display zone in which test values of the multiple test items are capable of being displayed;
an important test item reading section that accesses an important test item storage to read out important test items according to a test purpose, the important test item storage storing important test items selected for each test purpose from among the multiple test items; and
an image select information accepting section that accepts image select information indicating that at least a medical image is selected from among the multiple medical images displayed in the image display zone, wherein
the screen production unit retrieves a test purpose corresponding to the selected medical image through the test purpose reading section on the basis of the image select information, retrieves important test items through the important test item reading section on the basis of the retrieved test purpose, and lets merely test values of the important test items be displayed in the test value display zone on the basis of the retrieved important test items.

2. The medical test result display device set forth in claim 1, wherein
an injury or disease is designated in the test purpose.

3. The medical test result display device set forth in claim 1, wherein
when multiple medical images are selected on the test result display screen, the screen production unit retrieves respective test purposes of the multiple medical images and lets test values of those important test items corresponding to the retrieved test purposes be displayed in the test value display zone.

4. The medical test result display device set forth in claim 3, wherein
if there are a shared important test item and a not-shared important test item among those for the respective test purposes of the multiple medical images, test values of the shared the important test item are displayed distinguishably from test values of the not-shared the important test item in the test value display zone.

5. The medical test result display device set forth in claim 4, wherein
if there is a shared the important test item among those for the respective test purposes of the multiple medical images, merely test values of the shared the important test item are displayed in the test value display zone.

6. The medical test result display device set forth in claim 1, wherein
the medical images are displayed as size-reduced thumbnail images on the test result display screen.

7. The medical test result display device set forth in claim 6, wherein
when one of the thumbnail images is selected on the test result display screen, the medical image corresponding to the selected thumbnail image is displayed in an enlarged size that is larger than the thumbnail image.

8. The medical test result display device set forth in claim 7, wherein
when the medical image is displayed in the enlarged size, the screen production unit lets test values of the important test items which corresponds to the test purpose be displayed in a manner superimposed on the enlarged medical image.

9. The medical test result display device set forth in claim 1, further comprising:
a reference value information obtaining section that accesses a reference value storage to retrieve reference value information, the reference value storage storing reference value information that records a reference value range of test values for each of the test items; and
a comparing and determining section that compares the reference value range and the test values displayed in the test value display zone, to determine whether the test values are within the reference value range or not;
wherein the screen production unit lets test values within the reference value range and test values outside the reference value range distinguishably in the test value display zone.

10. The medical test result display device set forth in claim 1, wherein
the screen production unit produces a graph on the basis of test values obtained at clinical tests carried out on the patient at different times, the graph indicating temporal changes in test values of the same test item, and lets the graph be displayed in the test value display zone.

11. The medical test result display device set forth in claim 1, wherein
the screen production unit lets multiple medical images, which have been captured at different times, be displayed in a chronological arrangement on the test result display screen.

12. A method for operating a medical test result display device that displays test results of medical tests, comprising the steps of:
reading out medical images and test values of multiple test items from a medical image storage and a test results storage, respectively, wherein the medical image storage stores medical images captured at imaging tests carried out on a patient, each imaging test having at least a test purpose, and the test result storage stores numerical test values that represent test results of multiple test items included in clinical tests carried out on the patient;
reading at least a test purpose from a test purpose storage which stores respective test purposes of the imaging tests;
producing a test result display screen having an image display zone in which multiple medical images are capable of being displayed and a test value display zone in which test values of the multiple test items are capable of being displayed;
reading important test items from an important test item storage according to a test purpose, the important test item storage storing important test items selected for each test purpose from among the multiple test items; and
accepting image select information that indicates that at least a medical image is selected from among the multiple medical images displayed in the image display zone, wherein
the step of producing a test result display screen comprises the steps of retrieving a test purpose corresponding to the selected medical image on the basis of the image select information, retrieving important test items on the basis of the retrieved test purpose, and displaying merely test values of the important test items in the test value display zone on the basis of the retrieved important test items.
